(19) United States
(12) Patent Application Publication
(10) Pub. No.: US 2023/0390164 A1
(43) Pub. Date: Dec. 7, 2023

(54) Films and Oral Care Compositions Comprising Same

(71) Applicant: Colgate-Palmolive Company, New York, NY (US)

(72) Inventors: Dailin CHEN, Guangzhou (CN); Shaopeng XU, Guangzhou (CN); Didi WU, Guangzhou, Guangdong (CN)

(73) Assignee: Colgate-Palmolive Company, New York, NY (US)

(21) Appl. No.: 18/250,384

(22) PCT Filed: Oct. 29, 2021
(86) PCT No.: PCT/US2021/057248

§ 371 Date: Apr. 25, 2023

(30) Foreign Application Priority Data
Oct. 30, 2020 (CN) 202011200270.9

(51) Int. Cl. A61K 8/02; A61Q 11/00; A61K 8/24; A61K 8/73

(52) U.S. Cl. CPC A61K 8/0204; A61Q 11/00; A61K 8/24; A61K 8/731; A61K 2800/92; A61K 2800/30; A61K 2800/651

(57) ABSTRACT

Described herein are films comprising a particulate matter having a refractive index of from about 1.5 to about 2.0; a cellulosic material; and a polysaccharide; wherein the cellulosic material and the polysaccharide are present in a weight ratio of from about 2.2:1 to about 5:1; and wherein the film is substantially free of a titanium containing material. Compositions comprising same, and methods of using same are also described.

### CROSS-REFERENCE TO RELATED APPLICATION

This application claims the benefit of priority from Chinese Patent Application No. 202011200270.9, filed Oct. 30, 2020, the contents of which are hereby incorporated herein by reference in their entirety.

### BACKGROUND

Aesthetic effects have been acknowledged to play an important role in consumer acceptance of many products. In many cases ornamental effects have been used to distinguish particular products in the marketplace and identify products having particular distinct properties. In the dentifrice field, substantially clear dentifrice products such as toothpastes and gels which have incorporated therein contrasting colored flakes are known. Such flakes provide an aesthetic effect which the consumer finds pleasing and promotes the use of the dentifrice, particularly by children. Although such products have met with consumer approval, the art seeks to further improve the aesthetic effects as well as the cosmetic and therapeutic benefits of these products so as to encourage the use of dentifrices in practicing oral hygiene.

Further, there are certain ingredients, which are known to provide the desired opacity without compromising the toughness and/or stability of the films in a dentifrice. However, certain ingredients which are known to provide this benefit are associated with undesirable effects. As such, there remains a need for additional ingredients that provide the desired aesthetics, while not compromising the toughness and/or stability of the films in a dentifrice. Certain embodiments of the present inventions are designed to meet these, and other, ends.

### BRIEF SUMMARY

Some embodiments of the present invention provide films comprising: a polymer, a clay, a wax or a combination thereof; a natural gum; and particulate matter having a refractive index of from about 1.0 to about 2.5; wherein the a weight ratio of the polymer, clay, wax or combination thereof to the natural gum is from about 2.2:1 to about 5:1.

Further embodiments of the present invention provide oral care composition comprising: an orally acceptable carrier; and a plurality of films, or film fragments, wherein each film or film fragment comprises: a particulate matter having a refractive index of from about 1.5 to about 2.0; a cellulosic material; and a polysaccharide; wherein the cellulosic material and the polysaccharide are present in a weight ratio of from about 2.5:1; and wherein the film is substantially free of a titanium containing material.

In some embodiments, the present invention provides a flexible film comprising: a particulate matter having a refractive index of from about 1.5 to about 2.0; a cellulosic material; and a polysaccharide; wherein the cellulosic material and the polysaccharide are present in a weight ratio of from about 2.5:1; and wherein the film is substantially free of a titanium containing material.

Other embodiments of the present invention provide oral care compositions comprising a film as described herein.

Still further embodiments provide methods of using the films and compositions described herein.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before describing certain embodiments of the present invention in detail, it is to be understood that the invention is not limited in its application to the details of any particular embodiment shown. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but not limited to”. The term “including” should be interpreted as “including, but not limited to”.

The abbreviations and symbols as used herein, unless indicated otherwise, take their ordinary meaning. The abbreviation “wt. %” means percent by weight. The symbol “μL” refers to a microliter, or 10⁻⁶ liters. The symbol “°” refers to a degree, including a degree of an angle and degree of Celsius.

The term “about” when referring to a number means any number within a range of 10% of the number. For example, the phrase “about 0.050 wt. %” refers to a number between and including 0.04500 wt. % and 0.05500 wt. %.

As used throughout, ranges are used as shorthand for describing each and every value that is within the range. Any value within the range can be selected as the terminus of the range.

The term “mixture” is to be interpreted broadly. It refers to a mixture of ingredients. The mixture may be a solid, liquid, semisolid. If a mixture is a liquid, a mixture may be a solution, an emulsion, a dispersion, a mixture displaying the Tyndall effect, or any other homogeneous mixture. Under one embodiment, the mixture is shelf stable. When referring to a list of ingredients, unless specifically indicated otherwise, the term “mixture” refers to a mixture of the aforementioned ingredients with each other, a mixture of any of aforementioned ingredients with other ingredients that are not aforementioned, and to a mixture of several aforementioned ingredients with other ingredients that are not aforementioned. For example, the term “mixture” in the phrase “the fluoride source is selected from the group consisting of stannous fluoride, sodium fluoride, amine fluoride, sodium monofluorophosphate, and mixtures thereof” refers to any of the following: a mixture of stannous fluoride and sodium fluoride; or a mixture of stannous fluoride and amine fluoride; or a mixture of stannous fluoride and sodium monofluorophosphate; or a mixture of sodium fluoride and amine fluoride; or a mixture of sodium fluoride and sodium monofluorophosphate; or a mixture of amine fluoride, sodium monofluorophosphate; or a mixture of stannous fluoride and any other fluoride source; or a mixture of sodium fluoride and any other fluoride source; or a mixture of amine fluoride and any other fluoride source; or a mixture of sodium monofluorophosphate and any other fluoride source, and other combinations thereof.

Any member in a list of species that are used to exemplify or define a genus may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All references cited herein are hereby incorporated by reference in their entireties. In the event of a conflict in a definition in the present disclosure and that of a cited reference, the present disclosure controls.

The term “substantially clear” when used in to describe the present invention shall mean translucent or transparent. The term “dentifrice” shall include toothpastes and gels.

In some embodiments, the films described herein may comprise a particulate matter having a refractive index of from about 1.5 to 2; and a cellulosic material (e.g. hydropropylmethyl cellulose (“HPMC”)) and a starch, wherein the weight ratio of cellulosic material:starch is about 2.5:1 to 5:1. In some embodiments, the film may further comprise a colorant such a dye or pigment, a flavorant, sweetener and/or a therapeutic agent such as an antibacterial agent or a breath freshening agent.

The film thickness in size from 0.5 to 10 microns and preferably 2 to 3 microns. The dried film of the present invention is then cut or punched into shaped flakes having a particle size of 0.01 to 0.50 inches preferably 0.08 to 0.25 inches.

In some embodiments, the oral care composition further comprises an anti-malodor agent. In some embodiments, the additional anti-malodor compound is a known odor-controlling agent. In addition, other metal-containing compounds, such as those of copper, stannous, bismuth, strontium; and succulents or other ingredients which increase salivary flow, act to wash away odors, are useful in the compositions described herein. Certain strong citrus-based flavorants, odor-absorption complexes, which entrap or adsorb malodor molecules are also useful in the claimed compositions. For example, Ordenone® has the ability to encapsulate malodor molecules such as mercaptans, sulfides and amines within its structure, as disclosed in, for example, U.S. Pat. No. 6,664,254. Odor-controlling actives suitable also include, but are not limited to, enzymes that can interrupt the process by which odors are created. For example, odor-blocking enzymes such as arginine deiminase, can be effectively formulated in the compositions of the invention. Also, molecules that effectively inhibit the bacterial production of malodor molecules can be used to control odor, for example agents that interfere with the bacterial enzymes cysteine desulfhydrase and/or methionine gamma-lyase. Odor-controlling actives suitable for odor blocking or as odor blockers, include but are not limited to agents that act by oxidizing or otherwise chemically reacting with malodor molecules, including peroxides, perchlorites, and reactive molecules with activated double bonds.

In some embodiments, the oral care compositions of the present invention comprise a carrier. The carrier may include, but is not limited to water or other aqueous solvent systems. In some embodiments, the carrier is an orally acceptable carrier. In some embodiments, the orally acceptable carrier may further comprise a humectant. Possible humectants are ethanol, a polyhydric alcohol, which includes, but is not limited to glycerin, glycol, inositol, maltitol, mannitol, sorbitol, xylitol, propylene glycol, polypropylene glycol (PPG), polyethylene glycol (PEG) and mixtures thereof, or a saccharide, which includes, but is not limited to fructose, glucose, sucrose and mixtures of saccharides (e.g. honey).

In further embodiments, the oral care composition may further comprise an anti-bacterial agent. In other embodiments, the anti-bacterial agent is selected from: triclosan (5-chloro-2-(2,4-dichlorophenoxy)phenol); 8-hydroxyquinoline and salts thereof, a zinc or stannous ion source, such as zinc citrate, zinc sulphate, zinc glycinate, sodium zinc citrate, stannous fluoride, stannous monofluorophosphate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride,); bisguanides, such as chlorhexidine digluconate, hexetidine, octenidine, alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; or mixtures thereof.

In some embodiments, the anti-bacterial agent is present at a concentration of from about to about 3%, by weight, from about 0.05% to about 2%, by weight or from about to about 1.5% by weight.

In some embodiments, the oral care composition may further include anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants. anti-calculus agents, a source of fluoride ions, thickeners, an active agent for prevention or treatment of a condition or disorder of hard or soft tissue of the oral cavity, adhesive agents, a whitening agent and combinations thereof. It is understood that while general attributes of each of the above categories of materials may differ, there may be some common attributes and any given material may serve multiple purposes within two or more of such categories of materials. Preferably, the carrier is selected for compatibility with other ingredients of the composition.

Some embodiments of the present invention optionally comprise an amino acid. Suitable amino acids include, but are not limited to arginine, cysteine, leucine, isoleucine, lysine, alanine, asparagine, aspartate, phenylalanine, glutamate, glutamic acid, threonine, glutamine, tryptophan, glycine, valine, praline, serine, tyrosine, and histidine, and a combination of two or more thereof. The amino acids can include R- and L-forms and salt forms thereof. The amino acids (and salt forms thereof) can also include acid ester and/or fatty amide derivatives of the amino acid (e.g. ethyl lauroyl arginate hydrochloride (ELAH)).

Yet other embodiments of the present invention provide oral care compositions comprising an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Still further embodiments of the present invention comprise an anticalculus (tartar control) agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent is present in an amount of from about 0.1% to about 30%. The oral composition may include a mixture of different anticalculus agents. In one preferred embodiment, tetrasodium pyrophosphate (TSPP) and sodium tripolyphosphate (STPP) are used. In some embodiments, the anticalculus agent comprises TSPP at about 0.1% to about 5%, by weight. In other embodiments, the anticalculus agent comprises STPP at about 0.1% to about 10%, by weight.

Further embodiments of the present invention comprise various dentifrice ingredients to adjust the rheology and feel of the composition such as surface active agents, thickening or gelling agents, etc.

Some embodiments of the present invention provide oral care compositions comprising a stannous ion or a stannous ion source. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5%.

Some embodiments of the present invention provide an oral care composition comprising a surface active agent (surfactant). Suitable surfactants include without limitation water-soluble salts of C8-C20 alkyl sulfates, sulfonated monoglycerides of C8-C20 fatty acids, sarcosinates, taurates, sodium lauryl sulfate, sodium cocoyl monoglyceride sulfonate, sodium lauryl sarcosinate, sodium lauryl isoethionate, sodium laureth carboxylate and sodium dodecyl benzenesulfonate, and cocoamidopropyl betaine.

In some embodiments, the oral care composition comprises a thickening agent. Any orally acceptable thickening agent can be used, including without limitation carbomers, also known as carboxyvinyl polymers, carrageenans, also known as Irish moss and more particularly-carrageenan (iota-carrageenan), high molecular weight polyethylene glycols (such as Carbowax®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, and colloidal and/or fumed silica and mixtures of the same. One or more thickening agents are optionally present in a total amount of about 0.1% to about 90%, for example about 1% to about 50% or about 5% to about 35%.

Other embodiments of the present invention optionally comprise a flavorant, a sweetener, a colorant, a foam modulator, a mouth-feel agent and/or others additively may be included if desired, in the composition.

Yet other embodiments of the present invention comprise one or more additional active material(s), which is operable for the prevention or treatment of a condition or disorder of hard or soft tissue of the oral cavity, the prevention or treatment of a physiological disorder or condition, or to provide a cosmetic benefit. Examples of such further active ingredient comprise a sialagogue or saliva-stimulating agent, an antiplaque agent, an anti-inflammatory agent, and/or a desensitizing agent.

Adhesion enhancing agents can also be added to the oral care compositions which include but is not limited to waxes, inclusive of bees' wax, mineral oil, plastigel, (a blend of mineral oil and polyethylene), petrolatum, white petrolatum, shellac, versagel (blend of liquid paraffin, butene/ethylene/styrene hydrogenated copolymer) polyethylene waxes, microcrystalline waxes, polyisobutene, polyvinyl pyrrolidone/vinyl acetate copolymers, and insoluble polyacrylate copolymers.

Also effective as adhesion enhancing agents are liquid hydrophilic polymers including polyethylene glycols, nonionic polymers of ethylene oxide having the general formula: HOCH2 (CH2OCH2)n1CH2OH wherein n1 represents the average number of oxyethylene groups. Polyethylene glycols available from Dow Chemical are designated by a number such as 200, 300, 400, 600, 2000 which represents the approximate average molecular weight of the polymer, as well as nonionic block copolymer of ethylene oxide and propylene oxide of the formula: HO(C2H4O)a1(C3H6O)b1(C2H4O)c1H. The block copolymer is preferably chosen (with respect to a1, b1 and c1) such that the ethylene oxide constituent comprises from about 65 to about 75% by weight, of the copolymer molecule and the copolymer has an average molecular weight of from about 2,000 to about 15,000 with the copolymer being present in the liquid tooth whitening composition in such concentration that the composition is liquid at room temperatures.

A particularly desirable block copolymer for use in the practice of the present invention is available commercially from BASF and designated Pluraflo L1220 (PEG/PPG 116/66) which has an average molecular weight of about 9,800. The hydrophilic poly(ethylene oxide) block averages about 65% by weight of the polymer.

Synthetic anionic polycarboxylates may also be used in the oral compositions of the present invention as an efficacy enhancing agent for any antibacterial, antitartar or other active agent within the dentifrice composition. Such anionic polycarboxylates are generally employed in the form of their free acids or preferably partially or more preferably fully neutralized water soluble alkali metal (e.g. potassium and preferably sodium) or ammonium salts. Preferred are 1:4 to 4:1 copolymers of maleic anhydride or acid with another polymerizable ethylenically unsaturated monomer, preferably methylvinylether/maleic anhydride having a molecular weight (M.W.) of about 30,000 to about 1,800,000 most preferably about 30,000 to about 700,000. Examples of these copolymers are available from GAF Corporation under the trade name GANTREZ® (methylvinylether/maleic anhydride), e.g., AN 139 (M.W. 500,000), AN 119 (M.W. 250,000); S-97 Pharmaceutical Grade (M.W. 700,000), AN 169 (M.W. 1,200,000-1,800,000), and AN 179 (M.W. above 1,800,000); wherein the preferred copolymer is S-97 Pharmaceutical Grade (M.W. 700,000).

When present, the anionic polycarboxylates is employed in amounts effective to achieve the desired enhancement of the efficacy of any antibacterial, antitartar or other active agent within the oral composition. Generally, the anionic polycarboxylates is present within the oral composition from about 0.05% to about 4% by weight, preferably from about 0.5% to about 2.5% by weight.

Adhesion enhancing agents employed in compositions of various embodiments of the invention are present in an amount of from about 0 to about 20% by weight. Preferably, the adhesion enhancing agents are present in an amount of from about 2 to about 15% by weight.

Some embodiments of the present invention optionally comprise a whitening agent which includes, but is not limited to peroxide compounds such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof.

In some embodiments, a non-peroxide whitening agent may be included in the compositions of the present invention. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes. In some embodiments the whitening agent is separated from the aqueous carrier. In some embodiments the whitening agent is separated from the aqueous carrier by encapsulation of the whitening agent.

In certain embodiments, the composition comprises about 65%-99.9% of the carrier and further included ingredients, i.e. one or more of anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants, anti-calculus agents, a source of fluoride ions, thickeners, an active agent for prevention or treatment of a condition or disorder of hard or soft tissue of the oral cavity, a whitening agent and combinations thereof. In another embodiment of the composition, the composition comprises about 80%-99.5% of the carrier and further included ingredients. In another embodiment of the composition, the composition comprises about 90%-99% of the carrier and further included ingredients.

In some embodiments, an abrasive polishing material may also be included in the compositions of the present invention. In some embodiments, the abrasive polishing material may be any material which does not excessively abrade dentin. These include, for example, silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, and resinous abrasive materials such as particulate condensation products of urea and formaldehyde, and others such as disclosed by Cooley et al. in U.S. Pat. No. 3,070,510, Dec. 25, 1962, incorporated herein by reference. Mixtures of abrasives may also be used.

In some embodiments, the abrasive system comprises a silica. Under one embodiment, the silica functions as an abrasive agent. Under another embodiment the silica functions as a thickening agent. Under still another embodiment, the oral care composition comprises both an abrasive silica and a thickening silica.

Silicas suitable for use in the compositions of the present invention may be prepared by any means known or to be developed in the art, and may be surface modified, if desired, to increase the capacity of the particle to adhere to a tooth surface. Examples may be found in, e.g., U.S. Patent Application Publication No. 20070104660, the contents of which are incorporated herein by reference. In the embodiments, silica is present in the composition in an amount of 5% or greater by weight of the total composition. Alternatively, silica may be present in an amount of 5%, 6%, 7%, 8%, 9%, 10%, 15%, 20% or 25% by weight. In some cases, the film may include silica in an amount of about 0.5 to about 10 wt. %, based on the total weight of the film. For example, the film may include silica in an amount from about 0.5 to about 10 wt. %, about 0.5 to about 8 wt. %, about 0.5 to about 6 wt. %, about 0.5 to about 4 wt. %, about 0.5 to about 3 wt. %; about 1 to about 10 wt. %, about 1 to about 8 wt. %, about 1 to about 6 wt. %, about 1 to about 4 wt. %, about 1 to about 3 wt. %; about 2 to about 10 wt. %, about 2 to about 8 wt. %, about 2 to about 6 wt. %, about 2 to about 4 wt. %, about 2 to about 3 wt. %; about 3 to about 10 wt. %, about 3 to about 8 wt. %, about 3 to about 6 wt. %; about 4 to about 10 wt. %, about 4 to about 8 wt. %, or about 4 to about 6 wt. %, based on the total weight of the film composition.

In some embodiments, the silica comprises precipitated silica. Precipitated silica is an amorphous form of silica (silicon dioxide, SiO₂), which is a white, powdery material. Precipitated silica is produced by precipitation from a solution containing silicate salts. Under one embodiment, the production of precipitated silica starts with the reaction of an alkaline silicate solution with a mineral acid. Sulfuric acid and sodium silicate solutions are added simultaneously with agitation to water, followed by a precipitation carried out under alkaline conditions. The choice of agitation, duration of precipitation, the addition rate of reactants, their temperature and concentration, and pH can vary the properties of the silica. The formation of a gel stage is avoided by stirring at elevated temperatures. The resulting white precipitate is filtered, washed and dried in the manufacturing process.

Examples of silica include ZEODENT® 105-High, ZEODENT® 103, ZEODENT® 113, ZEODENT® 115, ZEODENT® 116, ZEODENT®117, ZEODENT® 120, ZEODENT® 124, ZEODENT® 153, ZEODENT® 163, ZEODENT® 165, ZEODENT® 167, ZEODENT® 168, ZEODENT® 203, ZEODENT®9175, available from Evonik; SYLODENT® 750 Silica, SYLODENT® 753 Silica, SYLODENT® 756 Silica, SYLOBLANC® 81 Silica, SYLODENT® SM 850C Silica, SYLOBLANC® 82 Silica, SYLODENT® SM 500T Silica, SYLODENT® SM 614T Silica, available from W. R. Grace; Tixosil® 63, Tixosil® 73, Tixosil® SoftClean™, Tixosil® 331, Tixosil® 43, available from Solvay; SORBOSIL AC33, SORBOSIL AC43, SORBOSIL BFG10, SORBOSIL BFG50, SORBOSIL BFG51, SORBOSIL BFG52, SORBOSIL BFG54, SORBOSIL CBT60S, SORBOSIL CBT70, SORBOSIL BFG100, available from PQ Corporation.

In certain embodiments, the silica comprises Sorbosil AC43 silica, available from PQ Corporation. In an embodiment, AC43 silica has properties including, an average particle size of 2.7 to 4.0 microns (as determined by MALVERN MASTERSIZER), a sieve residue of +45 μm, a moisture loss at 105° C. of 8.0% max, an ignition loss at 1000° C. of 14.0% max, and a pH of 5.5 to 7.5 in aqueous suspension.

In some embodiments, the thickener silica is a synthetic amorphous precipitated material of high surface area and internal pore volume to provide water absorption of about 50 ml or greater/20 grams of silica and oil absorption of about 200 ml or greater/100 g silica (per ASTM D281 method). Examples of thickener silicas which may be used are Zeodent® 165, Zeodent® 163 and Zeodent® 153; Aerosil® 200 and Sident® 22S (available from Evonik); Sylodent® 15 and Perkasil® SM 660 (available from W.R. Grace & Co.); MFIL®, MFIL® (available from Madhu Silica, India) and Tixocil 43B (available from Rhodia).

In other embodiments, the oral care compositions of the invention include silica particles with, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns.

Sodium bicarbonate can also be added to the oral care compositions of the present invention. Sodium bicarbonate, also known as baking soda, is a household product with a variety of uses including use in dentifrices and mouthrinses. It is a white powder that is soluble in water and unless stabilized, tends to release carbon dioxide in an aqueous system.

In some embodiments, the compositions of the present invention comprise a coloring agent. In some embodiments, the coloring agent comprises a pigment. As used herein, a “pigment” is a synthetic or natural water insoluble substance, which imparts color to another substance. In some embodiments, the pigments further enhance the whiteness of the teeth. As is known in the art, the visual perception of a white substance can be altered through the deposition of an optical brightener, a blue pigment, or a blue dye. This effect is commonly used in laundry detergent products to make white clothes appear “whiter” to the human eye. The same concept has been applied to tooth whitening. See PCT Publication No. WO 2015/099642 to Colgate-Palmolive Company, which is herein incorporated by reference in its entirety.

In other embodiments, the pigment is capable of reflecting sufficient light such that the treated tooth is perceivably whiter than its initial color. In some embodiments, the pigment may be colored such that its natural color is within the violet-red to green-blue color. More particularly, the pigment may be violet or blue, e.g., one of those listed in the Color Index International.

In some embodiments, the amount of pigment in the composition may be from 0.01 to 0.075 weight %, such as 0.05%. In other embodiments, the amount of pigment in the composition may be from 0.01 to 0.05 weight %, or from 0.03 to 0.05%, by weight based on the total amount of the composition. The pigment may be uniformly spread throughout the composition or may be dispersed in a second phase such as a stripe or other coextruded second phase. Such “dual phase” compositions have the advantage that the phases may be differently colored, presenting a more visually attractive product to the consumer.

In some embodiments, the coloring agent comprises a dye. As used herein, the term “dye” refers to an organic species, which is essentially water soluble in an aqueous medium in which the dye remains chemically stable. The dyes used with the whitening dentifrice composition of the present disclosure are generally food color additives presently certified under the Food Drug & Cosmetic Act for use in food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfonium-phenyl)-methylene}-[1-N-ethyl-N-p-sulfobenzyl)-.DELTA.-3,5-cyclohexadienim-ine], FD&C Blue No. 1 (disodium salt of dibenzyldiethyl-diaminotriphenylcarbinol trisulfonic acid anhydride), FD&C Blue No. 2 (sodium salt of disulfonic acid of indigotin) D&C Green No. 5, D&C Orange No. 5, D&C Red No. 21, D&C Red No. 22, D&C Red No. 27, D&C Red No. 28, D&C Red No. 30, D&C Red No. 40, D&C Yellow No. 10 and mixtures thereof in various proportions.

The amount of one or more of the dyes in the oral care composition may widely vary. For example, the amount of one or more of the dyes in the whitening dentifrice composition of the present disclosure may be from 0.02 to 2 weight %, or 0.02 to 1.5 weight %, or 0.02 to 1 weight %, or 0.02 to 0.5 weight %, 0.02 to 0.15 weight %, or 0.02 to 0.1 weight %, based on the total amount of the whitening dentifrice composition. In at least one embodiment, the one or more dyes may be disposed or dispersed uniformly throughout the whitening dentifrice composition. In another embodiment, the one or more dyes may be disposed or dispersed in different phases of the whitening dentifrice composition. For example, one or more of the dyes may be disposed or dispersed in a first phase (e.g., a hydrophobic phase) of the whitening dentifrice composition, and one or more of the remaining dyes, or no dye, may be disposed or dispersed in a second phase (e.g., a hydrophilic phase) of the whitening dentifrice composition.

In some embodiments, the surfactant is selected from water-soluble salts of C8-20 alkyl sulfates, sulfonated monoglycerides of C₈₋₂₀ fatty acids, sarcosinates, taurates, sodium lauryl sulfate, sodium cocoyl mono glyceride sulfonate, sodium lauryl sarcosinate, sodium lauryl isoethionate, sodium laureth carboxylate and sodium dodecyl benzenesulfonate, cocoamidopropyl betaine, and mixtures thereof.

Further examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of the last mentioned amides include N-lauryl sarcosine, and the sodium, potassium and ethanolamine salts of N-lauryl, N-myristoyl, or N-palmitoyl sarcosine. Others include, for example, nonanionic polyoxyethylene surfactants, such as Poloxamer 407, Steareth 30, Polysorbate 20, and castor oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides.

In some embodiments, the viscosity modifier is selected from methylcellulose, hydroxypropyl methyl cellulose, hydroxyethylpropyl cellulose, hydroxybutyl methyl cellulose, carboxymethyl cellulose, salts thereof, and mixtures thereof.

In other embodiments, the compositions of the invention may optionally comprise an additional orally acceptable thickening agent, selected from one or more of, without limitation, carbomers, also known as carboxyvinyl polymers, carrageenans, also known as Irish moss and more particularly carrageenan (iota-carrageenan), high molecular weight polyethylene glycols (such as CARBOWAX®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, and colloidal magnesium aluminum silicate and mixtures of the same. Optionally, such additional thickening agents are present in a total amount of about 0.1 wt. % to about 50 wt. %, for example about 0.1 wt. % to about 35 wt. % or about 1 wt. % to about 15 wt. %, based on the weight of the composition.

In some embodiments, the compositions of the present invention comprise at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like.

Still further embodiments provide compositions comprising a sweetener selected from: aspartame; acesulfame potassium; luo han guo (monk) fruit extract; neotame; saccharin; stevia; sucralose; xylitol; advantame; and mixtures thereof.

One or more sweeteners are optionally present in a total amount depending strongly on the particular sweetener(s) selected, but typically 0.005 wt. % to 5 wt. %, by total weight of the composition.

In some embodiments, the composition comprises a fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. In certain embodiments the fluoride ion source includes stannous fluoride, sodium fluoride, amine fluorides, sodium monofluorophosphate, as well as mixtures thereof. In certain embpdments, the oral care composition of the invention may also contain a source of fluoride ions or fluorine-providing ingredient in amounts sufficient to supply about 50 to about 5000 ppm fluoride ion, e.g., from about 100 to about 1000, from about 200 to about 500, or about 250 ppm fluoride ion. Fluoride ion sources may be added to the compositions of the invention at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts.

In some embodiments, the oral care composition is in a form selected from: a toothpaste; a liquid (e.g. a mouthwash or mouthrinse); a gel; a spray; or a composition which is applied to the teeth using a dental tray. In certain embodiments, the composition is in the form of a toothpaste. In some embodiments, the toothpaste is adapted to be applied to the teeth by brushing. In other embodiments, the oral care composition is in the form of an ingestible or non-ingestible solid (e.g. a tablet or bead).

The films described herein may be punched into various attractive shaped flakes such as hearts, stars, diamonds and circles.

In some embodiments, the cellulosic material comprises a low viscosity hydropropylmethyl cellulose (“HPMC”). When HPMC is used as the film forming agent it is preferred that the HPMC have a viscosity in the range of about 1 to about 40 millipascal seconds (mPa·s) as determined as a 2% by weight aqueous solution of the HPMC at a temperature of 20° C. using a Ubbelohde tube viscometer. Preferably, the HPMC has a viscosity of about 3 to about mPa·s at a temperature of 20° C. HPMC is available commercially from the Dow Chemical Company under the trade designation Methocel E5 LV. Methocel E5 LV is a USP grade, low viscosity HPMC having 29.1% methoxyl groups and 9% hydroxyproxyl group substitution. It is a white or off-white free-flowing dry powder. As a 2 wt. % solution in water as measured with an Ubbelohde tube viscometer it has a viscosity of 5.1 mPa·s at a temperature of 20° C.

Preferably, the cellulosic material comprises a hydroxyalkyl cellulose, such as those chosen from hydroxypropyl methyl cellulose (HPMC), hydroxypropyl cellulose, hyrdoxyethyl cellulose, methyl cellulose, carboxymethyl cellulose, and a mixture of two or more thereof. In some cases, the film comprises two or more cellulosic materials selected from hydroxypropyl methyl cellulose (HPMC), hydroxypropyl cellulose, hyrdoxyethyl cellulose, methyl cellulose, carboxymethyl cellulose, and a mixture of two or more thereof. For example, the film may comprise two or more hydroxypropyl methyl cellulose (HPMC) compounds. In at least one embodiment, the film includes a first hydroxypropyl methyl cellulose having a viscosity of 40-60 mpa·s. and a second hydroxypropyl methyl cellulose having a viscosity of 6.5-8.5 mpa·s. The weight ratio of the first hydroxypropyl methyl cellulose having a viscosity of 40-60 mpa·s to the second hydroxypropyl methyl cellulose having a viscosity of 6.5-8.5 mpa·s may be from about 1:1 to about 5:1, about 2:1 to about 4.5:1, about 2.5 to about 4:1, or about 3:1 to about 3.8:1.

Pregelatinized corn starch is available commercially. A preferred starch is available under the trade designation Cerestar Polar Tex-Instant 12640 from the Cerestar Company. This Cerestar starch is a pregelaterized, stabilized and crosslinked waxy maize starch. It is readily dispersible and swellable in cold water. In its dry form, it is a white free flowing powder with an average flake size no greater than 180 micrometers and 85% of the flakes are smaller than 75 micrometers. It has a bulk density of 44 lbs/ft³.

In some embodiments, the present invention provides a film for use in an oral care composition, comprising: a polymer, a clay a wax or a combination thereof; and particulate matter having a refractive index of from about 1.0 to about 2.5. The amount of polymer, a clay a wax or a combination thereof in the film may vary, but in some embodiments may be present in an amount from about 30 to about 70 wt. %, about 35 to about 70 wt. %, about 40 to about 70 wt. %, about 42 to about 70 wt. %, about 44 to about 70 wt. %, about 46 to about 70 wt. %, about 48 to about 70 wt. %; about 30 to about 65 wt. %, about 35 to about 65 wt. %, about 40 to about wt. %, about 42 to about 65 wt. %, about 44 to about 65 wt. %, about 46 to about 65 wt. %, about 48 to about 65 wt. %; about 30 to about 60 wt. %, about 35 to about 60 wt. %, about 40 to about 60 wt. %, about 42 to about 60 wt. %, about 44 to about 60 wt. %, about 46 to about 60 wt. %, about 48 to about 60 wt. %; about 30 to about 58 wt. %, about 35 to about 58 wt. %, about to about 58 wt. %, about 42 to about 58 wt. %, about 44 to about 58 wt. %, about 46 to about 58 wt. %, or about 48 to about 58 wt. %, based on the total weight of the film.

In some embodiments, the polymer is selected from: a water-soluble polymer; a water-insoluble polymer; a water-dispersible polymer; and a combination of two or more thereof. In other embodiments, the polymer comprises a cellulosic material; a vinyl polymer; an acrylate polymer; and a combination of two or more thereof.

In further embodiments, the cellulosic material comprises a hydroxyalkyl cellulose (e.g., hydroxypropyl methyl cellulose (HPMC), hydroxypropyl cellulose, hyrdoxyethyl cellulose, methyl cellulose, carboxymethyl cellulose, hydroxyethylcellulose, carboxymethylcellulose (carmellose) and salts thereof, and mixtures of two or more thereof). In certain embodiments, the vinyl polymer is selected from: polyvinylpyrrolidone; cross-linked polyvinyl pyrrolidone; polyvinylpyrrolidone-vinyl acetate copolymer; polyvinyl alcohol; a carboxy vinyl polymer; and a combination of two or more thereof.

Still further embodiments provide a film, wherein the polymer is selected from: a polyacrylic acid, a polyacrylate polymer, a cross-linked polyacrylate polymer, a cross-linked polyacrylic acid (e.g, Carbopol®), or a combination of two or more thereof.

In some embodiments, the film comprises a natural gum. The amount of natural gum (e.g., one or more starch) in the film may vary, but is typically present in an amount from about 5 to about 30 wt. %, based on the total weight of the film. For example, the film may have natural gum, such as one or more starch(es), in an amount of about 5 to about 30 wt. %, about 5 to about 26 wt. %, about 5 to about 22 wt. %, about 5 to about 20 wt. %, about 5 to about 18 wt. %, about 5 to about 17 wt. %, about 5 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 9 to about 30 wt. %, about 9 to about 26 wt. %, about 9 to about 22 wt. %, about 9 to about 20 wt. %, about 9 to about 18 wt. %, about 9 to about 17 wt. %, about 9 to about 16 wt. %; about 11 to about 30 wt. %, about 11 to about 26 wt. %, about 11 to about 22 wt. %, about 11 to about 20 wt. %, about 11 to about 18 wt. %, about 11 to about 17 wt. %, about 11 to about 16 wt. %; about 13 to about 30 wt. %, about 13 to about 26 wt. %, about 13 to about 22 wt. %, about 13 to about 20 wt. %, about 13 to about 18 wt. %, about 13 to about 17 wt. %, about 13 to about 16 wt. %; about 14 to about 30 wt. %, about 14 to about 26 wt. %, about 14 to about 22 wt. %, about 14 to about 20 wt. %, about 14 to about 18 wt. %, about 14 to about 17 wt. %, or about 14 to about 16 wt. %, based on the total weight of the film.

In other embodiments, the natural gum is selected from: sodium alginate; carrageenan; xanthan gum; gum acacia; arabic gum; guar gum; pullulan; agar; chitin; chitosan; pectin; karaya gum; locust bean gum; tragacanth; a starch (e.g., maltodextrin, amylose, high amylose starch, corn starch, potato starch, rice starch, tapioca starch, pea starch, sweet potato starch, barley starch, wheat starch, waxy corn starch, modified starch (e.g. hydroxypropylated high amylose starch), dextrin, levan, elsinan; gluten); and a combination of two or more thereof. The corn starch may be a modified corn starch, modified amylose starch, or a combination thereof.

In some embodiments, the particulate matter is selected from: a zinc compound; a calcium compound; a stannous compound; a stannic compound; a tin compound; and a combination of two or more thereof. In further embodiments, the particulate matter is selected from: zinc oxide; calcium pyrophosphate; dicalcium phosphate dihydrate; calcium carbonate; tin oxide; stannic oxide; and a combination of two or more thereof. In some embodiments, the particulate material comprises calcium pyrophosphate.

In certain embodiments, the particulate matter has a refractive index from about 1.1 to about 2.4, optionally from about 1.2 to about 2.3, from about 1.3 to about 2.2, from about 1.4 to about 2.1, from about 1.5 to about 2.0, from about 1.5 to about 1.9, from about 1.5 to about 1.8, from about 1.5 to about 1.7, or about 1.5 to about 1.6.

In some embodiments, the polymer comprises a cellulosic material. In further embodiments, the film comprises a cellulosic material and a starch. In other embodiments, the polymer and starch are present in a weight ratio of from about 2.5:1 to about 5:1.

In some embodiments, the cellulosic material comprises hydroxypropyl methylcellulose. The film may include cellulosic material in an amount ranging from about 10 to about 70 wt. %, based on the total weight of the film. In some cases, the amount of cellulosic material present in the film is from about 30 to about 70 wt. %, about 35 to about 70 wt. %, about 40 to about 70 wt. %, about 42 to about 70 wt. %, about 44 to about 70 wt. %, about 46 to about 70 wt. %, about 48 to about 70 wt. %; about 30 to about 65 wt. %, about 35 to about 65 wt. %, about 40 to about 65 wt. %, about 42 to about 65 wt. %, about 44 to about 65 wt. %, about 46 to about 65 wt. %, about 48 to about 65 wt. %; about 30 to about 60 wt. %, about 35 to about 60 wt. %, about 40 to about 60 wt. %, about 42 to about 60 wt. %, about 44 to about 60 wt. %, about 46 to about 60 wt. %, about 48 to about 60 wt. %; about 30 to about 58 wt. %, about 35 to about 58 wt. %, about 40 to about 58 wt. %, about 42 to about 58 wt. %, about 44 to about 58 wt. %, about 46 to about 58 wt. %, or about 48 to about 58 wt. %, based on the total weight of the film.

In further cases, the amount of cellulosic material present in the film is from about 10 to about 50 wt. %, about 10 to about 45 wt. %, about 10 to about 40 wt. %, about 10 to about 35 wt. %, about 10 to about 30 wt. %, about 10 to about 28 wt. %; about 15 to about 50 wt. %, about to about 45 wt. %, about 15 to about 40 wt. %, about 15 to about 35 wt. %, about 15 to about 30 wt. %, about 15 to about 28 wt. %; about 20 to about 50 wt. %, about 20 to about 45 wt. %, about to about 40 wt. %, about 20 to about 35 wt. %, about 20 to about 30 wt. %, about 20 to about 28 wt. %; about 22 to about 50 wt. %, about 22 to about 45 wt. %, about 22 to about 40 wt. %, about 22 to about 35 wt. %, about 22 to about 30 wt. %, about 22 to about 28 wt. %; about 24 to about 50 wt. %, about 24 to about 45 wt. %, about 24 to about 40 wt. %, about 24 to about 35 wt. %, about 24 to about 30 wt. %, about 24 to about 28 wt. %, based on the total weight of the film.

In some embodiments, the cellulosic material is present in an amount of from about 1 wt. % to about 20 wt. %, based on the total weight of the film. In other embodiments, the cellulosic material is present in an amount of from about 5 wt. % to about 15 wt. %, based on the total weight of the film. In further embodiments, the cellulosic material is present in an amount of from about 7.5 wt. % to about 12.5 wt. %, based on the total weight of the film. Still further embodiments provide a film wherein the cellulosic material is present in an amount of from about 8 wt. % to about 10 wt. %, based on the total weight of the film. In some embodiments, the cellulosic material is present in an amount of about 9 wt. %, based on the total weight of the film.

In certain embodiments, the starch is present in an amount of from about 1 wt. % to about wt. %, based on the total weight of the film. In some embodiments, the starch is present in an amount of from about 2 wt. % to about 4.5 wt. %, based on the total weight of the film. In further embodiments, the starch is present in an amount of from about 3.5 wt. % to about 4.25 wt. %, based on the total weight of the film. Still further embodiments provide a film, wherein the starch is present in an amount of from about 3.75 wt. % to about 4 wt. %, based on the total weight of the film.

The film may have a composition with a weight ratio of polymer, clay, wax or combination thereof to natural gum from about 1:1 to about 5:1. In some embodiments, the film has a weight ratio of polymer, clay, wax or combination thereof to natural gum from about 1:1 to about 5:1, about 1.5:1 to about 5:1, about 2:1 to about 5:1, about 2.25:1 to about 5:1, about 2.5:1 to about 5:1, about 2.75:1 to about 5:1, about 3:1 to about 5:1, about 3.2:1 to about 5:1, about 3.4:1 to about 5:1; about 2.5:1 to about 4.7:1, about 2.75:1 to about 4.7:1, about 3:1 to about 4.7:1, about 3.2:1 to about 4.7:1, about 3.4:1 to about 4.7:1; about 2.5:1 to about 4.4:1, about 2.75:1 to about 4.4:1, about 3:1 to about 4.4:1, about 3.2:1 to about 4.4:1, about 3.4:1 to about 4.4:1; about 2.5:1 to about 4.1:1, about 2.75:1 to about 4.1:1, about 3:1 to about 4.1:1, about 3.2:1 to about 4.1:1, about 3.4:1 to about 4.1:1; about 2.5:1 to about 3.9:1, about 2.75:1 to about 3.9:1, about 3:1 to about 3.9:1, about 3.2:1 to about 3.9:1, about 3.4:1 to about 3.9:1; about 2.5:1 to about 3.7:1, about 2.75:1 to about 3.7:1, about 3:1 to about 3.7:1, about 3.2:1 to about 3.7:1, or about 3.4:1 to about 3.7:1, including any ranges thereof. In at least one embodiment, the film has a weight ratio of polymer, clay, wax or combination thereof to natural gum of about 3.6:1.

Additionally or alternatively, the film may have a composition with a weight ratio of cellulosic material to natural gum (e.g., starch) from about 1:1 to about 5:1. In some embodiments, the film has a weight ratio of cellulosic material to natural gum (e.g., starch) from about 1:1 to about 5:1, about 1.5:1 to about 5:1, about 2:1 to about 5:1, about 2.25:1 to about 5:1, about 2.5:1 to about 5:1, about 2.75:1 to about 5:1, about 3:1 to about 5:1, about 3.2:1 to about 5:1, about 3.4:1 to about 5:1; about 2.5:1 to about 4.7:1, about 2.75:1 to about 4.7:1, about 3:1 to about 4.7:1, about 3.2:1 to about 4.7:1, about 3.4:1 to about 4.7:1; about 2.5:1 to about 4.4:1, about 2.75:1 to about 4.4:1, about 3:1 to about 4.4:1, about 3.2:1 to about 4.4:1, about 3.4:1 to about 4.4:1; about 2.5:1 to about 4.1:1, about 2.75:1 to about 4.1:1, about 3:1 to about 4.1:1, about 3.2:1 to about 4.1:1, about 3.4:1 to about 4.1:1; about 2.5:1 to about 3.9:1, about 2.75:1 to about 3.9:1, about 3:1 to about 3.9:1, about 3.2:1 to about 3.9:1, about 3.4:1 to about 3.9:1; about 2.5:1 to about 3.7:1, about 2.75:1 to about 3.7:1, about 3:1 to about 3.7:1, about 3.2:1 to about 3.7:1, or about 3.4:1 to about 3.7:1, including any ranges thereof. In at least one embodiment, the film has a weight ratio of cellulosic material to natural gum (e.g., starch) of about 3.6:1.

The film may have a composition with a weight ratio of polymer, clay, wax or combination thereof to natural gum from about 2.2:1 to about 3.2:1. In some embodiments, the film has a weight ratio of polymer, clay, wax or combination thereof to natural gum from about 2.2:1 to about 3.2:1, about 2.2:1 to about 3:1, about 2.2:1 to about 2.8:1, about 2.2:1 to about 2.6:1, about 2.2:1 to about 2.5:1; about 2.3:1 to about 3.2:1, about 2.3:1 to about 3:1, about 2.3:1 to about 2.8:1, about 2.3:1 to about 2.6:1, about 2.3:1 to about 2.5:1; about 2.4:1 to about 3.2:1, about 2.4:1 to about 3:1, about 2.4:1 to about 2.8:1, about 2.4:1 to about 2.6:1, or about 2.4:1 to about 2.5:1, including any ranges thereof. In at least one embodiment, the film has a weight ratio of polymer, clay, wax or combination thereof to natural gum of about 2.5:1.

Additionally or alternatively, the film may have a composition with a weight ratio of cellulosic material to natural gum (e.g., starch) from about 2.2:1 to about 3.2:1. In some embodiments, the film has a weight ratio of cellulosic material to natural gum (e.g., starch) from about 2.2:1 to about 3.2:1, about 2.2:1 to about 3:1, about 2.2:1 to about 2.8:1, about 2.2:1 to about 2.6:1, about 2.2:1 to about 2.5:1; about 2.3:1 to about 3.2:1, about 2.3:1 to about 3:1, about 2.3:1 to about 2.8:1, about 2.3:1 to about 2.6:1, about 2.3:1 to about 2.5:1; about 2.4:1 to about 3.2:1, about 2.4:1 to about 3:1, about 2.4:1 to about 2.8:1, about 2.4:1 to about 2.6:1, or about 2.4:1 to about 2.5:1, including any ranges thereof. In at least one embodiment, the film has a weight ratio of cellulosic material to natural gum (e.g., starch) of about 2.5:1.

The film may include a particulate material in an amount from about 1 to about 70 wt. %, based on the total weight of the film. For instance, in some cases, the particulate material may be present in the film in an amount from about 10 to about 30 wt. %, about 10 to about 27 wt. %, about 10 to about 25 wt. %, about 10 to about 23 wt. %, about 10 to about 21 wt. %, about 10 to about 20 wt. %; about 12 to about 30 wt. %, about 12 to about 27 wt. %, about 12 to about 25 wt. %, about 12 to about 23 wt. %, about 12 to about 21 wt. %, about 12 to about 20 wt. %; about 14 to about 30 wt. %, about 14 to about 27 wt. %, about 14 to about 25 wt. %, about 14 to about 23 wt. %, about 14 to about 21 wt. %, about 14 to about 20 wt. %; about 16 to about 30 wt. %, about 16 to about 27 wt. %, about 16 to about 25 wt. %, about 16 to about 23 wt. %, about 16 to about 21 wt. %, or about 16 to about 20 wt. %, based on the total weight of the film. The film may include a lower amount of particulate matter, for example, depending on the particulate matter incorporated therein. For example, the film may include particulate matter in an amount from about 1 to about 10 wt. %, about 1 to about 8 wt. %, about 1 to about 6 wt. %, about 1 to about 4 wt. %, about 1 to about 3 wt. %; about 2 to about 10 wt. %, about 2 to about 8 wt. %, about 2 to about 6 wt. %, about 2 to about 4 wt. %, about 2 to about 3 wt. %; about 3 to about 10 wt. %, about 3 to about 8 wt. %, about 3 to about 6 wt. %; about 4 to about 10 wt. %, about 4 to about 8 wt. %, or about 4 to about 6 wt. %, based on the total weight of the film composition.

In further cases, the particulate material may be present in the film in an amount from about 20 to about 70 wt. %, about 25 to about 70 wt. %, about 30 to about 70 wt. %, about 35 to about 70 wt. %, about 40 to about 70 wt. %, about 42 to about 70 wt. %, about 44 to about 70 wt. %, about 46 to about 70 wt. %, about 48 to about 70 wt. %; about 20 to about 65 wt. %, about to about 65 wt. %, about 30 to about 65 wt. %, about 35 to about 65 wt. %, about 40 to about 65 wt. %, about 42 to about 65 wt. %, about 44 to about 65 wt. %, about 46 to about 65 wt. %, about 48 to about 65 wt. %; about 20 to about 60 wt. %, about 25 to about 60 wt. %, about 30 to about 60 wt. %, about 35 to about 60 wt. %, about 40 to about 60 wt. %, about 42 to about 60 wt. %, about 44 to about 60 wt. %, about 46 to about 60 wt. %, about 48 to about 60 wt. %; about 20 to about 55 wt. %, about 25 to about 55 wt. %, about 30 to about 55 wt. %, about 35 to about 55 wt. %, about to about 55 wt. %, about 42 to about 55 wt. %, about 44 to about 55 wt. %, about 46 to about 55 wt. %, or about 48 to about 55 wt. %, about 20 to about 50 wt. %, about 25 to about 50 wt. %, about 30 to about 50 wt. %, about 35 to about 50 wt. %, about 40 to about 50 wt. %, about 42 to about 50 wt. %, about 44 to about 50 wt. %, about 46 to about 50 wt. %, or about 48 to about 50 wt. %; about 20 to about 47 wt. %, about 25 to about 47 wt. %, about 30 to about 47 wt. %, about to about 47 wt. %, about 40 to about 47 wt. %, about 42 to about 47 wt. %, based on the total weight of the film.

In some embodiments, the present invention provides a film comprising from about 0.1 wt. % to about 20 wt. % of the particulate material. In other embodiments, the present invention provides a film comprising from about 1 wt. % to about 18 wt. %, from about 5 wt. % to about 16 wt. %, or about 10 wt. % to about 15 wt. %, of the particulate material. Still further embodiments of the present invention provide a film comprising about 15 wt. % of the particulate material.

In some embodiments, the film is substantially free of titanium containing materials. For example, the film may have about 3 wt. % or less, 2 wt. % or less, 1 wt. % or less, 0.5 wt. % or less, or 0.1 wt. % or less of a titanium containing material, based on the total weight of the film. In other embodiments, the film is free of titanium containing materials. In certain embodiments, the titanium containing material is selected from titanium dioxide and titanium coated mica.

In some embodiments, the present invention provides a film comprising a plasticizer. In other embodiments, the present invention provides a plasticizer selected from: glycerol; a phthalate derivative; propylene glycol; a low molecular weight polyethylene glycol; and a combination of two or more thereof.

Nonionic surfactants may be useful in the certain embodiments of the films disclosed herein. Examples of nonionic surfactants include compounds produced by the condensation of alkylene oxides (especially ethylene oxide) with an organic hydrophobic compound, which may be aliphatic or alkylaromatic in nature. One group of surfactants is known as “ethoxamers”. These include condensation products of ethylene oxide with fatty acids, fatty alcohols, fatty amides, polyhydric alcohols, (e.g., sorbitan monostearate) and the like. “Polysorbates” is the name given to a class of nonionic surfactants prepared by ethoxylating the free hydroxyls of sorbitan-fatty acid esters. They are commercially available, for example as the TWEEN™ surfactants of ICI, US Inc. Non-limiting examples include Polysorbate 20 (polyoxyethylene 20 sorbitan monolaurate, TWEEN™ 20) and Polysorbate 80 (polyoxyethylene 20 sorbitan mono-oleate, TWEEN™ 80). In certain embodiments, polysorbates include those with about 20 to 60 moles of ethylene oxide per mole of sorbitan ester.

Nonionic surfactants are optionally present in embodiments of this invention at amounts of about 0.1% to about 10 wt. %, based on the total weight of the film. In some embodiments, the film includes one or more nonionic surfactants in an amount of about 0.1 to about 8 wt. %, about 0.1 to about 6 wt. %, about 0.1 to about 5 wt. %, about 0.1 to about 4 wt. %; about 1 to about 8 wt. %, about 1 to about 6 wt. %, about 1 to about 5 wt. %, about 1 to about 4 wt. %; 2 to about 8 wt. %, about 2 to about 6 wt. %, about 2 to about 5 wt. %, or about 2 to about 4 wt. %, based on the total weight of the film.

The film may, in some cases, include a flavorant in amount from about 1 to about 20 wt. %, about 1 to about 18 wt. %, about 1 to about 16 wt. %, about 1 to about 14 wt. %, about 1 to about 12 wt. %; about 3 to about 20 wt. %, about 3 to about 18 wt. %, about 3 to about 16 wt. %, about 3 to about 14 wt. %, about 3 to about 12 wt. %; about 5 to about 20 wt. %, about 5 to about 18 wt. %, about 5 to about 16 wt. %, about 5 to about 14 wt. %, about 5 to about 12 wt. %; about 7 to about 20 wt. %, about 7 to about 18 wt. %, about 7 to about 16 wt. %, about 7 to about 14 wt. %, about 7 to about 12 wt. %; about 9 to about 20 wt. %, about 9 to about 18 wt. %, about 9 to about 16 wt. %, about 9 to about 14 wt. %, or about 9 to about 12 wt. %, based on the total weight of the film composition.

The flavorant may be chosen from menthone, isopulegol, N-ethyl p-menthanecarboxamide, N,2,3-trimethyl-2-isopropylbutanamide, ethyl 2-(p-menthane-3-carboxamido) acetate, N-(4-menthoxyphenyl)-p-menthane-3-carboxamide, menthyl lactate, menthone glycerine acetal, mono-menthyl succinate, mono-menthyl glutarate, 0-menthyl glycerine, menthyl-N,N-dimethylsuccinamate, 2-sec-butylcyclohexanone, and N-(4-cyanomethylphenyl)-p-menthanecarboxamide, menthol, and a combination of two or more thereof. The flavorant may preferably be a menthol, such as I-menthol.

In some embodiments, the present invention provides a film having a breaking strength of greater than about 750 psi (5,171 kPa). The present invention provides, a film described herein having a breaking strength of greater than about 1,000 psi (6,894 kPa), 1,100 psi (7,584 kPa), greater than about 1,250 psi (8,618 kPa) or greater than about 1,500 psi (10,342 kPa). In various embodiments, the breaking strength of the film is about 750 psi (5,171 kPa) to about 5,000 psi (34,470 kPa) or about 750 psi (5,171 kPa) to about 2,900 psi (19,995 kPa).

In certain embodiments, the present invention provides a film having a thickness of from about 0.01 mm to about 0.1 mm. In other embodiments, the present invention provides a film having a thickness of from about 0.25 mm to about 0.75 mm. In further embodiments, the present invention provides a film having a thickness of about 0.05 mm.

In some embodiments, the present invention provides an oral care composition comprising any one of the films described herein. In some embodiments, the present invention provides an oral care composition comprising a film that remains substantially undissolved after about 1 month at 40° C. In some embodiments, the present invention provides an oral care composition comprising a film that remains substantially undissolved after about 2 months at 40° C.

In other embodiments, the present invention provides an oral care composition comprising a film that remains substantially undissolved after about 1 month at 60° C. In further embodiments, the present invention provides an oral care composition comprising a film that remains substantially undissolved after about 2 months at 60° C.

Further embodiments provide an oral care composition comprising: an orally acceptable carrier; and a plurality of films, or film fragments, wherein each film or film fragment comprises: a particulate matter having a refractive index of from about 1.5 to about 2.0; a cellulosic material; and a polysaccharide; wherein the cellulosic material and the polysaccharide are present in a weight ratio of from about 2.2:1 to about 5:1; and wherein the film is substantially free of a titanium containing material.

Other embodiments of the present invention provide a method of cleaning an oral cavity surface of a mammalian subject, comprising administering any one of the films or compositions described herein to an oral cavity surface of a subject in need thereof. Further embodiments of the present invention provide methods that further comprise the step of rinsing the oral cavity.

### EXEMPLARY EMBODIMENTS OF THE INVENTION

In accordance with certain embodiments, provided is a film for use in an oral care composition, the film comprising:

An oral care film comprising:
a polymer, a clay, a wax or a combination thereof, wherein the polymer, the clay, the wax, or a combination thereof is, preferably, present in an amount from about 10 to about 70 wt. %, about 35 to about 70 wt. %, about 40 to about 70 wt. %, about 42 to about 70 wt. %, about 44 to about 70 wt. %, about 46 to about 70 wt. %, about 48 to about 70 wt. %; about 30 to about 65 wt. %, about 35 to about 65 wt. %, about 40 to about 65 wt. %, about 42 to about 65 wt. %, about 44 to about 65 wt. %, about 46 to about 65 wt. %, about 48 to about 65 wt. %; about 30 to about 60 wt. %, about 35 to about 60 wt. %, about 40 to about 60 wt. %, about 42 to about 60 wt. %, about 44 to about 60 wt. %, about 46 to about 60 wt. %, about 48 to about 60 wt. %; about 30 to about 58 wt. %, about 35 to about 58 wt. %, about 40 to about 58 wt. %, about 42 to about 58 wt. %, about 44 to about 58 wt. %, about 46 to about 58 wt. %, or about 48 to about 58 wt. %, based on the total weight of the film;
a natural gum, preferably, in an amount from about 5 to about 30 wt. %, about 5 to about 26 wt. %, about 5 to about 22 wt. %, about 5 to about 20 wt. %, about 5 to about 18 wt. %, about 5 to about 17 wt. %, about 5 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 9 to about 30 wt. %, about 9 to about 26 wt. %, about 9 to about 22 wt. %, about 9 to about 20 wt. %, about 9 to about 18 wt. %, about 9 to about 17 wt. %, about 9 to about 16 wt. %; about 11 to about 30 wt. %, about 11 to about 26 wt. %, about 11 to about 22 wt. %, about 11 to about 20 wt. %, about 11 to about 18 wt. %, about 11 to about 17 wt. %, about 11 to about 16 wt. %; about 13 to about 30 wt. %, about 13 to about 26 wt. %, about 13 to about 22 wt. %, about 13 to about 20 wt. %, about 13 to about 18 wt. %, about 13 to about 17 wt. %, about 13 to about 16 wt. %; about 14 to about 30 wt. %, about 14 to about 26 wt. %, about 14 to about 22 wt. %, about 14 to about 20 wt. %, about 14 to about 18 wt. %, about 14 to about 17 wt. %, or about 14 to about 16 wt. %, based on the total weight of the film,
   wherein a weight ratio of the polymer, clay, wax or combination thereof to the natural gum is from about 1:1 to about 5:1, about 1.5:1 to about 5:1, about 2:1 to about 5:1, about 2.25:1 to about 5:1, about 2.5:1 to about 5:1, about 2.75:1 to about 5:1, about 3:1 to about 5:1, about 3.2:1 to about 5:1, about 3.4:1 to about 5:1; about 2.5:1 to about 4.7:1, about 2.75:1 to about 4.7:1, about 3:1 to about 4.7:1, about 3.2:1 to about 4.7:1, about 3.4:1 to about 4.7:1; about 2.5:1 to about 4.4:1, about 2.75:1 to about 4.4:1, about 3:1 to about 4.4:1, about 3.2:1 to about 4.4:1, about 3.4:1 to about 4.4:1; about 2.5:1 to about 4.1:1, about 2.75:1 to about 4.1:1, about 3:1 to about 4.1:1, about 3.2:1 to about 4.1:1, about 3.4:1 to about 4.1:1; about 2.5:1 to about 3.9:1, about 2.75:1 to about 3.9:1, about 3:1 to about 3.9:1, about 3.2:1 to about 3.9:1, about 3.4:1 to about 3.9:1; about 2.5:1 to about 3.7:1, about 2.75:1 to about 3.7:1, about 3:1 to about 3.7:1, about 3.2:1 to about 3.7:1, or about 3.4:1 to about 3.7:1, including any ranges thereof;
particulate having a refractive index of from about 1.0 to about 2.5, the particulate comprising stannic oxide, dicalcium phosphate, calcium carbonate, or a combination of two or more thereof, the particulate, preferably, present in an amount from about 1 to about 30, such as, e.g., about 10 to about 30 wt. %, about 10 to about 27 wt. %, about 10 to about 25 wt. %, about 10 to about 23 wt. %, about 10 to about 21 wt. %, about 10 to about 20 wt. %; about 12 to about 30 wt. %, about 12 to about 27 wt. %, about 12 to about 25 wt. %, about 12 to about 23 wt. %, about 12 to about 21 wt. %, about 12 to about 20 wt. %; about 14 to about 30 wt. %, about 14 to about 27 wt. %, about 14 to about 25 wt. %, about 14 to about 23 wt. %, about 14 to about 21 wt. %, about 14 to about 20 wt. %; about 16 to about 30 wt. %, about 16 to about 27 wt. %, about 16 to about 25 wt. %, about 16 to about 23 wt. %, about 16 to about 21 wt. %, about 16 to about 20 wt. %, about 1 to about 10 wt. %, about 1 to about 8 wt. %, about 1 to about 6 wt. %, about 1 to about 4 wt. %, about 1 to about 3 wt. %; about 2 to about 10 wt. %, about 2 to about 8 wt. %, about 2 to about 6 wt. %, about 2 to about 4 wt. %, about 2 to about 3 wt. %; about 3 to about 10 wt. %, about 3 to about 8 wt. %, about 3 to about 6 wt. %; about 4 to about 10 wt. %, about 4 to about 8 wt. %, or about 4 to about 6 wt. % based on the total weight of the film; and
optionally, an amount of menthol or a derivative thereof, preferably in an amount from about 1 to about 20 wt. %, about 1 to about 18 wt. %, about 1 to about 16 wt. %, about 1 to about 14 wt. %, about 1 to about 12 wt. %; about 3 to about 20 wt. %, about 3 to about 18 wt. %, about 3 to about 16 wt. %, about 3 to about 14 wt. %, about 3 to about 12 wt. %; about 5 to about 20 wt. %, about 5 to about 18 wt. %, about 5 to about 16 wt. %, about 5 to about 14 wt. %, about 5 to about 12 wt. %; about 7 to about 20 wt. %, about 7 to about 18 wt. %, about 7 to about 16 wt. %, about 7 to about 14 wt. %, about 7 to about 12 wt. %; about 9 to about 20 wt. %, about 9 to about 18 wt. %, about 9 to about 16 wt. %, about 9 to about 14 wt. %, or about 9 to about 12 wt. %, based on the total weight of the film composition,
wherein the film is substantially free of titanium dioxide, and all weight percentages are based on the total weight of the oral care film.

According with another embodiment, provided is a film for use in an oral care composition, the film comprising:

An oral care film comprising:
a polymer, a clay, a wax or a combination thereof, the polymer, the clay, the wax, or a combination thereof is preferably present in an amount from about 10 to about 50 wt. %, about 10 to about 45 wt. %, about 10 to about 40 wt. %, about 10 to about 35 wt. %, about 10 to about 30 wt. %, about 10 to about 28 wt. %; about 15 to about 50 wt. %, about 15 to about 45 wt. %, about 15 to about 40 wt. %, about 15 to about 35 wt. %, about 15 to about 30 wt. %, about 15 to about 28 wt. %; about 20 to about 50 wt. %, about 20 to about 45 wt. %, about 20 to about 40 wt. %, about 20 to about 35 wt. %, about 20 to about 30 wt. %, about 20 to about 28 wt. %; about 22 to about 50 wt. %, about 22 to about 45 wt. %, about 22 to about 40 wt. %, about 22 to about 35 wt. %, about 22 to about 30 wt. %, about 22 to about 28 wt. %; about 24 to about 50 wt. %, about 24 to about 45 wt. %, about 24 to about 40 wt. %, about 24 to about 35 wt. %, about 24 to about 30 wt. %, or about 24 to about 28 wt. %, based on the total weight of the film;
a natural gum, preferably, in an amount of about 5 to about 30 wt. %, about 5 to about 26 wt. %, about 5 to about 22 wt. %, about 5 to about 20 wt. %, about 5 to about 18 wt. %, about 5 to about 17 wt. %, about 5 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 7 to about 30 wt. %, about 7 to about 26 wt. %, about 7 to about 22 wt. %, about 7 to about 20 wt. %, about 7 to about 18 wt. %, about 7 to about 17 wt. %, about 7 to about 16 wt. %; about 9 to about 30 wt. %, about 9 to about 26 wt. %, about 9 to about 22 wt. %, about 9 to about 20 wt. %, about 9 to about 18 wt. %, about 9 to about 17 wt. %, about 9 to about 16 wt. %; about 11 to about 30 wt. %, about 11 to about 26 wt. %, about 11 to about 22 wt. %, about 11 to about 20 wt. %, about 11 to about 18 wt. %, about 11 to about 17 wt. %, about 11 to about 16 wt. %; about 13 to about 30 wt. %, about 13 to about 26 wt. %, about 13 to about 22 wt. %, about 13 to about 20 wt. %, about 13 to about 18 wt. %, about 13 to about 17 wt. %, about 13 to about 16 wt. %; about 14 to about 30 wt. %, about 14 to about 26 wt. %, about 14 to about 22 wt. %, about 14 to about 20 wt. %, about 14 to about 18 wt. %, about 14 to about 17 wt. %, or about 14 to about 16 wt. %, based on the total weight of the film,
   wherein a weight ratio of the polymer, clay, wax or combination thereof to the natural gum is from about 2.2:1 to about 3.2:1, about 2.2:1 to about 3:1, about 2.2:1 to about 2.8:1, about 2.2:1 to about 2.6:1, about 2.2:1 to about 2.5:1; about 2.3:1 to about 3.2:1, about 2.3:1 to about 3:1, about 2.3:1 to about 2.8:1, about 2.3:1 to about 2.6:1, about 2.3:1 to about 2.5:1; about 2.4:1 to about 3.2:1, about 2.4:1 to about 3:1, about 2.4:1 to about 2.8:1, about 2.4:1 to about 2.6:1, or about 2.4:1 to about 2.5:1, including any ranges thereof; and
particulate having a refractive index of from about 1.0 to about 2.5, the particulate comprising calcium pyrophosphate, wherein the particulate is preferably present in an amount of about 20 to about 70 wt. %, about 25 to about 70 wt. %, about 30 to about 70 wt. %, about 35 to about 70 wt. %, about 40 to about 70 wt. %, about 42 to about 70 wt. %, about 44 to about 70 wt. %, about 46 to about 70 wt. %, about 48 to about 70 wt. %; about 20 to about 65 wt. %, about 25 to about 65 wt. %, about 30 to about 65 wt. %, about 35 to about 65 wt. %, about 40 to about 65 wt. %, about 42 to about 65 wt. %, about 44 to about 65 wt. %, about 46 to about 65 wt. %, about 48 to about 65 wt. %; about 20 to about 60 wt. %, about 25 to about 60 wt. %, about 30 to about 60 wt. %, about 35 to about 60 wt. %, about 40 to about 60 wt. %, about 42 to about 60 wt. %, about 44 to about 60 wt. %, about 46 to about 60 wt. %, about 48 to about 60 wt. %; about 20 to about 55 wt. %, about 25 to about 55 wt. %, about 30 to about 55 wt. %, about 35 to about 55 wt. %, about 40 to about 55 wt. %, about 42 to about 55 wt. %, about 44 to about 55 wt. %, about 46 to about 55 wt. %, or about 48 to about 55 wt. %, about 20 to about 50 wt. %, about 25 to about 50 wt. %, about 30 to about 50 wt. %, about 35 to about 50 wt. %, about 40 to about 50 wt. %, about 42 to about 50 wt. %, about 44 to about 50 wt. %, about 46 to about 50 wt. %, or about 48 to about 50 wt. %; about 20 to about 47 wt. %, about 25 to about 47 wt. %, about 30 to about 47 wt. %, about 35 to about 47 wt. %, about 40 to about 47 wt. %, or about 42 to about 47 wt. %, based on the total weight of the film; and
optionally, an amount of menthol or a derivative thereof, preferably in an amount of about 1 to about 20 wt. %, about 1 to about 18 wt. %, about 1 to about 16 wt. %, about 1 to about 14 wt. %, about 1 to about 12 wt. %; about 3 to about 20 wt. %, about 3 to about 18 wt. %, about 3 to about 16 wt. %, about 3 to about 14 wt. %, about 3 to about 12 wt. %; about 5 to about 20 wt. %, about 5 to about 18 wt. %, about 5 to about 16 wt. %, about 5 to about 14 wt. %, about 5 to about 12 wt. %; about 7 to about 20 wt. %, about 7 to about 18 wt. %, about 7 to about 16 wt. %, about 7 to about 14 wt. %, about 7 to about 12 wt. %; about 9 to about 20 wt. %, about 9 to about 18 wt. %, about 9 to about 16 wt. %, about 9 to about 14 wt. %, or about 9 to about 12 wt. %, based on the total weight of the film composition,
wherein the film is substantially free of titanium dioxide, and all weight percentages are based on the total weight of the oral care film.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

Various experiments were conducted to evaluate various properties of the films of the present invention, for example: opacity; toughness; and stability of the films in a toothpaste. The methods for evaluating the films and the results generated from these experiments are described below.

Opacity: comometer was used to measure L*a*b* of the film and calculate ΔW by below formula: W*=((a*−0)2+(b*−0)2+(L*−100)2)½; ΔW=W(film)−W(control). The higher AW score, the better film opacity. Significant difference statistically is considered to define whether the opacity is pass or fail. Neutral means the AW of the sample is lower than control but the appearance in the toothpaste is still good.

Toughness: Toughness is mainly defined by the process of stretch out the slurry of the films on the platform. If the film is too viscous to be stretched out, then it is failed on toughness. Meanwhile, whether the films can be formed homogeneous after drying is also used to evaluate the toughness. Only the films that is smooth and unbroken can be treated as pass on toughness.

Stability in Toothpaste: The films were cut into square strips and then applied in the clear toothpastes at a dosage of 0.12%. Then the toothpastes were placed in oven at 60C for one month to observe the performance of strips. Some of the strips dissolve in the tooth paste after aging with a bad stability in the toothpaste. Whereas some of the strips still stay well in the toothpaste after aging and are mentioned acceptable in the form below.

### Example 1

Films were prepared having similar compositions, with the specific calcium compound and the amount thereof being varied between the prepared Films. Each of the films were prepared by producing a slurry composition and then drying the slurry composition to remove water. After drying the respective slurry compositions, the films were evaluated for opacity, toughness, and stability in a toothpaste composition. For example, as seen in Table 1, below, three films were produced with various amounts of natural calcium carbonate and evaluated for opacity, toughness, and stability in toothpaste. Table 2 shows the evaluation of five films produced with various amounts of dicalcium phosphate dehydrate. Table 3 shows the evaluation of three films produced with various amounts of calcium pyrophosphate. The weight percentages shown in Tables 1-3 are based on the total weight of the slurry compositions used to prepare such films.

TABLE 1
Natural Calcium Carbonate

| | | | Stability in |
|---|---|---|---|
| Dosage | | | Toothpaste |
| (wt. %) | Opacity | Toughness | (“TP”) |
| 0.96% | Fail | Pass | Dissolved |
| 3% | Fail | Pass | Dissolved |
| 10% | Neutral | Neutral | Acceptable |

TABLE 2
Dicalcium Phosphate Dihydrate

| | | | Stability in |
|---|---|---|---|
| Dosage | | | Toothpaste |
| (wt. %) | Opacity | Toughness | (“TP”) |
| 0.96% | Neutral | Pass | Dissolved |
| 2% | Pass | Pass | Dissolved |
| 3% | Pass | Pass | Dissolved |
| 5% | Pass | Pass | Dissolved |
| 10% | Pass | Fail | n/a |

TABLE 3
Calcium Pyrophosphate

| | | | Stability in |
|---|---|---|---|
| Dosage | | | Toothpaste |
| (wt. %) | Opacity | Toughness | (“TP”) |
| 0.96% | Fail | Pass | Dissolved |
| 7% | Neutral | Pass | Acceptable |
| 10% | Pass | Fail | n/a |

### Example 2

Five films were prepared having similar compositions, but with the weight ratio of HPMC to starch being different. Each of the films was produced by preparing a slurry composition and forming a film therefrom by drying the slurry composition to remove water. The film forming properties of the five films were then evaluated. The weight percentages shown in Tables 4 for Comparative Examples I-IV and Example I are based on the total weight of the slurry composition used to prepare such films.

TABLE 4
HPMC/Starch Ratio Evaluation

| | Comp. | Comp. | Comp. | | Comp. |
|---|---|---|---|---|---|
| | Ex. I | Ex. II | Ex. III | Ex. I | Ex. IV |
| | Wt. % | | | | |
| HPMC | 3.13 | 6.5 | 8.13 | 9.38 | 12.5 |
| Starch | 3.8 | 3.84 | 3.84 | 3.84 | 3.84 |
| Calcium | 15 | 15 | 15 | 15 | 15 |
| pyrophosphate | | | | | |
| HPMC/Starch | 0.8 | 1.7 | 2.1 | 2.5 | 3.3 |
| Ratio | | | | | |
| Film-forming | No | No | No | Yes | No |

### Example 3

Three films were prepared to assess the opacity, toughness, and stability of films produced with dicalcium phosphate dihydrate (“Dical”) and calcium pyrophosphate (“Cal Pyro”). Each of the films was produced by preparing a slurry composition and forming a film therefrom by drying the slurry composition to remove water. The weight percentages shown in Tables 5 are based on the total weight of the slurry composition used to prepare such films.

TABLE 5
Dical and Cal Pyro Evaluation

| | 10% Dical | 15% Dical | 15% Cal Pyro |
|---|---|---|---|
| HPMC (wt. %) | 9.38 | 9.38 | 9.38 |
| Film Opacity | High | High | High |
| Film toughness | High | High | Acceptable |
| Film Stability | Dissolved | Dissolved | Not Dissolved |

### Example 4

Five films were prepared to assess the opacity, toughness, and stability of films produced with various amounts of calcium pyrophosphate (“Cal Pyro”). Each of the films was produced by preparing a slurry composition and forming a film therefrom by drying the slurry composition to remove water. The weight percentages for the films shown in Tables 6 are based on the total weight of the slurry composition used to prepare such films.

TABLE 6
Cal Pyro Evaluation

| Dosage | Opacity | Toughness | Stability in TP |
|---|---|---|---|
| 0.96% | Fail | Pass | Dissolved |
| 7% | Neutral | Pass | Acceptable |
| 10% | Neutral | Pass | Acceptable |
| 15% | Pass | Pass | Acceptable |
| 18% | Pass | Fail | n/a |

### Example 5

Four films were prepared to assess the opacity, toughness, and stability of films produced with various amounts of stannic oxide. Each of the films was produced by preparing a slurry composition and forming a film therefrom by drying the slurry composition to remove water. The weight percentages for the films shown in Tables 7 are based on the total weight of the slurry composition used to prepare such films.

TABLE 7
Stannic Oxide Level Evaluation

| | | | 1 Month @ |
|---|---|---|---|
| Dosage | Opacity | Toughness | 60° C. |
| 0.5% | Fail | Pass | n/a |
| 0.96% | Pass | Pass | Pass |
| 3% | Pass | Pass | Pass |
| 5% | Pass | Pass | Pass |

### Example 6

An exemplary film (Ex. II) was prepared in accordance with aspects of the invention. Five comparative films (Comp. Ex. V-IX) were prepared with formulations similar to Ex. II, except that Comp. Ex. V-IX have different weight ratios of HPMC to starch. The weight percentages for the formulations in Table 8 are based on the total weight of the respective film's composition.

TABLE 8

| | Comp. | Comp. | Comp. | | Comp. | Comp. |
|---|---|---|---|---|---|---|
| | Ex. V | Ex. VI | Ex. VII | Ex. II | Ex. VIII | Ex. IX |
| US INCI Name | (wt. %) | (wt. %) | (wt. %) | (wt. %) | (wt. %) | (wt. %) |
| HPMC | 10.9 | 19.7 | 24.2 | 26.9 | 32.9 | 38 |
| Corn Starch | 13.45 | 12.13 | 11.45 | 11.04 | 10.13 | 9.36 |
| Total weight ratio of | 0.8 | 1.6 | 2.1 | 2.4 | 3.3 | 4.1 |
| cellulosic material to | | | | | | |
| starch | | | | | | |
| Propylene glycol | 6.7 | 6.1 | 5.7 | 5.5 | 5.1 | 4.7 |
| Polysorbate 80 | 1.7 | 1.5 | 1.43 | 1.4 | 1.3 | 1.2 |
| Calcium pyrophosphate | 52.5 | 47.3 | 44.7 | 43.1 | 39.5 | 36.5 |
| Menthol | 14.7 | 13.3 | 12.5 | 12.1 | 11.1 | 10.2 |

Comparative Compositions V and VI failed to form a film. Comparative Composition VII was brittle and contained cracks. Comparative Compositions VIII and IX had a rough surface and a spongy shape, respectively. Surprisingly, Example Composition II formed a film that was smooth and had no cracks.

### Example 7

An exemplary film (Ex. III) was prepared having the formulation shown in Table 9 in accordance with aspects of the invention. The weight percentages for the formulation of Ex. III are based on the total weight of the film's composition.

TABLE 9

| | Comp. Ex. III |
|---|---|
| US INCI Name | (wt. %) |
| HPMC | 50.2 |
| Corn Starch | 14.1 |
| Total weight ratio of | 3.6 |
| cellulosic material to | |
| starch | |
| Propylene glycol | 7.7 |
| Polysorbate 80 | 1.8 |
| Tin Oxide | 11 |
| Menthol | 15.4 |

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1-60. (canceled)

61. A film for use in an oral care composition, comprising:
a polymer, a clay, a wax or a combination thereof;
a natural gum; and
a particulate having a refractive index of from about 1.0 to about 2.5;
wherein the film is substantially free of titanium containing materials; and
wherein the weight ratio of the polymer, clay, wax or combination thereof to the natural gum is from about 2.2:1 to about 5:1, and all weight percentages are based on the total weight of the film.

62. The film according to claim 1 comprising:
from about 30 to about 70 wt. % of the polymer, the clay, the wax or the combination thereof;
from about 5 to about 30 wt. % of the natural gum;
wherein a weight ratio of the polymer, clay, wax or combination thereof to the natural gum is from about 2.5:1 to about 5:1;
a particulate having a refractive index of from about 1 to about 2.5, the particulate selected from: stannic oxide; dicalcium phosphate; calcium carbonate; and a combination of two or more thereof; and
optionally, an amount of menthol or a derivative thereof;
wherein the film is substantially free of titanium dioxide, and all weight percentages are based on the total weight of the oral care film.

63. The film according to claim 62, wherein the polymer, the clay, the wax or the combination thereof comprises a cellulosic material.

64. The film according to claim 63, wherein the cellulosic material comprises two or more cellulosic materials selected from: hydroxypropyl methyl cellulose; hydroxypropyl cellulose; hyrdoxyethyl cellulose; methyl cellulose; and carboxymethyl cellulose.

65. The film according to claim 63, wherein the cellulosic material comprises a first hydroxypropyl methyl cellulose having a viscosity of 40-60 mpa·s. and a second hydroxypropyl methyl cellulose having a viscosity of 6.5-8.5 mpa·s.

66. The film according to claim 61, wherein the polymer comprises a vinyl polymer selected from polyvinylpyrrolidone; cross-linked polyvinyl pyrrolidone; polyvinylpyrrolidone-vinyl acetate copolymer; polyvinyl alcohol; a carboxy vinyl polymer; and a combination of two or more thereof.

67. The film according to claim 61, wherein the polymer is selected from: a polyacrylic acid; a polyacrylate polymer; a cross-linked polyacrylate polymer; a cross-linked polyacrylic acid; and a combination of two or more thereof.

68. The film according to claim 61, wherein the natural gum is selected from: sodium alginate; carrageenan; xanthan gum; gum acacia; gum arabic; guar gum; pullulan; agar; chitin; chitosan; pectin; karaya gum; locust bean gum; tragacanth; maltodextrin; amylose; high amylose starch; corn starch; potato starch; rice starch; tapioca starch; pea starch; sweet potato starch; barley starch; wheat starch; waxy corn starch; hydroxypropylated high amylose starch; dextrin; levan; elsinan; gluten; and a combination of two or more thereof.

69. The film according to claim 61, wherein the particulate comprises a zinc compound.

70. The film according to claim 61, wherein the particulate comprises calcium pyrophosphate.

71. The film according to claim 61, wherein the film is free of titanium dioxide and free of titanium coated mica.

72. The film according to claim 61, further comprising a plasticizer selected from: glycerol; a phthalate derivative; propylene glycol; a low molecular weight polyethylene glycol; and a combination of two or more thereof.

73. The film according to claim 61, wherein the film has a breaking strength greater than 750 psi.

74. The film according to claim 61, wherein the film has a thickness of from about 0.01 mm to about 0.1 mm.

75. The oral care composition according to claim 61, wherein the film remains substantially undissolved after about 2 months at 40° C.

76. The oral care composition according to claim 61, wherein the film remains substantially undissolved after about 1 month at 60° C.

77. An oral care film comprising:
from about 10 to about 50 wt. % of a polymer, a clay, a wax or a combination thereof;
from about 5 to about 30 wt. % of a natural gum,
wherein a weight ratio of the polymer, clay, wax or combination thereof to the natural gum is from about 2.2:1 to about 3.2:1; and
a particulate having a refractive index of from about 1.0 to about 2.5, the particulate comprising calcium pyrophosphate; and
optionally, an amount of menthol or a derivative thereof,
wherein the film is substantially free of titanium dioxide, and all weight percentages are based on the total weight of the oral care film.

78. An oral care composition comprising:
an orally acceptable carrier; and
a plurality of films, or film fragments, wherein each film or film fragment comprises:
a particulate matter having a refractive index of from about 1.5 to about 2.0;
a cellulosic material; and
a polysaccharide;
wherein the cellulosic material and the polysaccharide are present in a weight ratio of from about 2.2:1 to about 5:1; and
wherein the film is substantially free of a titanium containing material.

79. The oral care composition according to claim 78, wherein the particulate material comprises calcium pyrophosphate.

80. The oral care composition according to claim 78, wherein the cellulosic material a cellulosic material selected from: hydroxypropyl methyl cellulose; hydroxypropyl cellulose; hyrdoxyethyl cellulose; methyl cellulose; carboxymethyl cellulose; and a combination of two or more thereof.
